(19) United States
(12) Patent Application Publication
(10) Pub. No.: US 2011/0300529 A1
(43) Pub. Date: Dec. 8, 2011

(54) DETECTION OF IFI16 IN BODY FLUIDS

(75) Inventors: Michele Mondini, Ivrea (IT); Santo Landolfo, Torino (IT); Marisa Gariglio, Poirino (IT); Silvia Costa, Tollegno (IT); Erica Miraglia, Torino (IT); Francesca Gugliesi, Torino (IT)

(73) Assignee: NoToPharm srl, Novara (IT)

(21) Appl. No.: 13/139,318

(22) PCT Filed: Dec. 14, 2009
(86) PCT No.: PCT/EP2009/067128

§ 371 Date: Aug. 26, 2011

Related U.S. Application Data
(60) Provisional application No. 61/122,091, filed on Dec. 12, 2008.

(51) Int. Cl. G01N 33/53; G01N 21/64; C12Q 1/70; G01N 21/76

(52) U.S. Cl. 435/5; 435/7.92; 435/7.1; 436/501

(57) ABSTRACT

The present invention relates to methods for the qualitative and/or quantitative determination of interferon inducible protein 16 (IFI 16) in an extracellular form.

The present invention relates to methods for the qualitative and/or quantitative determination of interferon inducible protein 16 (IFI16) in an extracellular form.

The interferon-inducible protein IFI16 belongs to the family of Interferon (IFN)-activatable genes designated HIN200 in the human and Ifi200 in the murine species. It was demonstrated that IFI16 is a nuclear phosphoprotein which participates in the inhibition of cell cycle progression, and involvement of the inflammatory process.

Immunohistochemical analysis of IFI16 expression in normal human tissues revealed that it is expressed in a highly restricted pattern in selected cells within certain organs. IFI16 is expressed in CD34+ myeloid precursor cells and remains strongly expressed within monocyte precursors, peripheral blood monocytes, and throughout lymphoid development. IFI16 was found in epithelial cells of the skin, gastrointestinal tract, urogenital tract and glands and ducts of breast tissues. In addition, all vascular endothelial cells from both blood and lymph vessels strongly expressed IFI16.

IFI16 expression can be induced by interferons, as well as by an array of cytokines and differentiating agents. In HL-60 cells, IFI16 was induced by dimethylsulfoxide, retinoic acid, and 1.25 dihydroxy vitamin D3. IFI16 is stimulated in HUVEC endothelial cells by oxidative stress and by pro-inflammatory molecules such as TNF-α and interleukin-1β (IL-1β).

Several lines of evidence link the interferons (IFNs) to autoimmune disorders, in particular to SSc and Systemic Lupus Erythematosus (SLE). Many observations suggest a role for IFI16 in systemic autoimmune diseases, in which chronic inflammation is involved. IFI16 expression was greatly increased and ubiquitously detected in all layers of the epidermis in the lesional skin from both SSc and SLE patients. Furthermore, the dermal inflammatory infiltrate showed IFI16 positive staining, indicating that it is expressed at a high level in lymphocytes, fibroblasts and EC.

Further, autoantibodies against IFI16 were found in the sera of patients affected by Systemic Sclerosis (SSc), Systemic Lupus Erythematosus (SLE) and Sjögren's Syndrome (SjS).

A possible use of IFI16 as molecular marker, however, appeared to be limited to solid tissue samples, since previously described results show that the protein IFI16 has an intracellular localization. Therefore, previous detection procedures were carried out with solid tissue samples from patients. Taking solid tissue samples from lesional tissue has, however, disadvantageous effects for the patients and is difficult. Moreover, since it is difficult to obtain solid tissue samples from healthy subjects, the use of IFI16 as molecular marker has been limited by the difficulty to compare IFI16 expression in lesional vs healthy samples.

Until now, experiments to evaluate possible IFI16 dysregulation in pathological conditions were carried out on solid tissue samples or cultured cells and to extracts from tissues, circulating or cultured cells, since it was assumed in the prior art that IFI16 is a protein which is active within the cell and is located in the nucleolus and the nucleoplasm of human cells (as shown by both confocal microscopy and immunoblotting of nuclear proteins).

As a mechanism for the generation of anti-IFI16 autoantibodies, it was hypothesized in the literature that IFI16 could be released from dying cells (Mondini M. et al., 2006). The process of cell death is recognized as a possible source of several autoantigens, and the most accepted mechanism of release is the relocalization of the nuclear antigens in apoptotic blebs (thus restricted within the membrane barrier) and/or their exposure to immune effectors at cell-membrane levels. This phenomenon has been demonstrated for several autoantigens, including Ro/SSa, La/SSB and oxidized nuclear antigens (i.e. LeFeber et al., 1984; Casciola-Rosen L. et al., 1994; Saegusa et al. 2002). An association of extracellular IFI16 protein with pathological conditions has not been published.

Based on this state of knowledge, however, it could not have been expected that a detectable amount of extracellular form of IFI16 might exist in the extracellular environment and that such amount of an extracellular IFI16 would be associated with pathological conditions.

Surprisingly, however, the present inventors have found that extracellular IFI16 may be determined in appropriate samples by simple and direct analytic methods.

Thus, in a first aspect, the present invention refers to an in vitro method for determining extracellular interferon inducible protein 16 (IFI16) in a sample.

In particular, the present inventors found that the presence and/or an increased amount of extracellular IFI16 is indicative for a pathological condition. Thus, the present invention is suitable as a diagnostic method for any pathologic condition associated with increased presence of IFI16 compared to a normal, i.e. healthy, control.

The term “sample” as used herein refers to a biological sample obtained from the purpose of evaluation in vitro. In the methods of the present invention, the sample, which is tested for extracellular IFI16, is preferably a body fluid sample, e.g. blood, plasma, serum, urine, saliva etc. Alternatively, the supernatant of a tissue sample or the supernatant of a cell culture sample may be tested. Preferably, the sample preparation does not involve any lysis of cells, particularly no lysis of cells known to express IFI16, e.g. epithelial or endothelial cells. The sample may be derived from a mammalian organism, e.g. a human organism or a mammalian, e.g. human cell culture according to known methods.

In the context of the invention, the term “body fluids” comprises all kinds of body fluids, optionally diluted or concentrated. Examples are blood, serum, plasma, amniotic fluid, brain/spinal cord fluid, liquor, cerebrospinal fluid, sputum, throat and pharynx secretions and other mucous membrane secretions, synovial fluids, ascites, tear fluid, lymph fluid and urine. Preferably, the body fluid is blood, plasma or serum.

According to the present invention, the detection of IFI16 may include the detection of full-length IFI16, or fragments thereof, particularly fragments, which have immunological activity of IFI16, e.g. which are immunologically detectable, and which may be produced by cleavage, e.g. enzymatic cleavage, and which may be indicative of pathological conditions, e.g. inflammatory diseases.

The term “determination” and/or “detection” comprises a qualitative or a quantitative determination of extracellular IFI16 in a sample. In a preferred embodiment, the determination is a qualitative or semi-quantitative determination, i.e. it is determined whether IFI16 is present or absent or it is determined whether the concentration of IFI16 is above or below a cut-off value. As the skilled artisan will appreciate, in a Yes-(presence) or No-(absence) assay, the assay sensitivity is usually set to match the cut-off value. A cut-off value can, for example, be determined from the testing of a group of healthy individuals. Preferably, the cut-off is set to result in a specificity of 90%, also preferred the cut-off is set to result in a specificity of 95%, or also preferred the cut-off is set to result in a specificity of 98%. Presence of a value above the cut-off value can, for example, be indicative for the presence of pathological conditions, in particular for example autoimmune and/or inflammatory disorders. In a further preferred embodiment, the determination is a quantitative determination. In this embodiment, the concentration of extracellular IFI16 is correlated to underlying diagnostic question like, e.g. stage of disease, disease progression or response to therapy.

Preferably, the determination of extracellular IFI16 comprises:
(a) contacting the sample with at least one receptor, which specifically binds to IFI16, and
(b) detecting the specific binding of the receptor to IFI16.

According to the invention, the term “specific binding” describes a specific interaction between a receptor and IFI16 or a fragment thereof. The specific interaction can be characterised with a “key-lock-principle”. The receptor and IFI16 have structures or motifs which fit with each other specifically, as e.g. an antigenic determinant (epitope) which interacts with the antigen binding site of an antibody.

The receptor, which specifically binds to IFI16, has at least an affinity of 10⁶l/mol for IFI16, preferably of at least 10⁷ l/mol for IFI16, more preferably an affinity of at least 10⁸ l/mol or also preferred of at least 10⁹l/mol for IFI16. As the skilled artisan will appreciate the term specific is used in particular to indicate that other biomolecules present in the sample do not significantly bind to the receptor specific for IFI16. Preferably, the level of binding to a biomolecule other than the target IFI16 results in a binding affinity which is at most only 10% or less, only 5% or less only 2% or less or only 1% or less of the affinity to the target IFI16, respectively. A preferred receptor, which specifically binds to IFI16, will fulfill both the above minimum criteria for affinity as well as for specificity.

In a further preferred embodiment of the method according to the invention, the detection of a specific binding of IFI16 with the first receptor in step (a), the sample is contacted with a second receptor for IFI16, which binds with an epitope of IFI16 and which is accessible after binding of the first receptor with IFI16.

In an especially preferred embodiment, the method of the invention involves the use of at least two receptors which specifically bind to IFI16, wherein one receptor is a detectable receptor and the other receptor is immobilized on a solid phase or carries a group, which allows immobilization to a solid phase, e.g. via specific binding to a complementary member of a binding pair on the surface.

This preferred embodiment relates, for example, to methods taking advantage of the mechanistic principle of the sandwich ELISA. This principle is generally known to the person skilled in the art. Furthermore, a corresponding method is described in Examples 1 and 3.

The detectable receptor may carry a detectable labelling group. Methods allowing labelling of a receptor are known in the art. Alternatively, the detectable receptor group may be specifically recognized by means of another, third receptor comprising a detectable labelling group.

Preferred examples of such labelling groups are radioactive or fluorescent labelling groups.

Further preferred labelling groups comprise enzyme labelling groups, e.g. alkaline phosphatase, peroxidase, [beta]-galactosidase, glucoamylase, urease and chloramphenicol acetyltransferase. Appropriate examples and the use of necessary substrates for the detection by means of enzymatic reactions are known to the person skilled in the art, amongst others from the package leaflet of commercially available detection kits. Such commercially available kits often contain antibodies which recognise the antibodies of specific species, e.g. anti-mouse, and to which enzymes emitting signals are coupled. Thus, corresponding antibodies are examples of the third receptor, which recognise a specific labelling of the second receptor, that is its Fc part.

The receptor may be selected from the group consisting of peptides, polypeptides, low-molecular substances, antibodies or fragments or derivatives thereof and aptamers. In a preferred embodiment, the receptor is an antibody or an antigen binding fragment thereof.

The term “peptide” usually refers to amino acid chains with up to 30 amino acids.

The term “polypeptide” refers to peptides which usually comprise more than 30 amino acids and includes proteins.

The term “low-molecular substance” or small molecule refers to molecules which are of lower molecular complexity than the macromolecules defined above. In the literature, the term “low-molecular substance” is not used in a uniform manner. In WO 89/03041 and WO 89/03042, molecules with a molecular mass of up to 7000 g/mol are described as small molecules. Usually, however, molecular masses between 50 and 3000 g/mol, more often, however, between 75 and 2000 g/mol and mostly in the range between 100 and 1000 g/mol are stated. Examples are known to the person skilled in the art from the documents (WO86/02736, WO97/31269, U.S. Pat. No. 5,928,868, U.S. Pat. No. 5,242,902, U.S. Pat. No. 5,468,651, U.S. Pat. No. 5,547,853, U.S. Pat. No. 5,616,562, U.S. Pat. No. 5,641,690, U.S. Pat. No. 4,956,303 and U.S. Pat. No. 5,928,643. Low-molecular substances can be of organic or inorganic nature.

According to the invention, the term “antibody” comprises polyclonal sera as well as monoclonal antibodies.

Monoclonal antibodies and methods for the production thereof are known to the person skilled in the art. These are based on a method first described by Köhler and Milstein (1975). This method is described in detail in, amongst others, the laboratory manual by Harlow and Lane (Antibodies, A laboratory manual; Cold Spring Harbor Laboratory; (1988); Chapter 6). By this definition, bispecific antibodies, synthetic antibodies and fragments or derivative of these antibodies are also comprised. These comprise fragments such as Fab, Fv or scFv and chemically modified derivatives of these antibodies or antibody fragments.

Aptamers are, in principle, known to the person skilled in the art from prior art.

Preferably, the method according to the invention is an ELISA, an EIA or a RIA. Appropriate methods are, in principle, known to the person skilled in the art from Harlow and Lane, loc. cit. and Rehm, loc. cit.

The surprising result that IFI16, so far known as an intracellular protein, is found in extracellular environment makes it possible to analyse IFI16 in the culture supernatant of tissue samples, samples of body fluids or samples of cell culture supernatants. By means of the method according to the invention, IFI16 can be detected in a simple and fast manner and, thus, serves as diagnostic parameter.

An association of extracellular IFI16 with pathological condition has not been published yet. Thus, there is no suggestion that a determination of extracellular IFI16 in body fluids would allow assessment of a pathological condition. Surprisingly, it was found in the present invention that a determination of the presence and/or amount of extracellular IFI16 in body fluid samples allows the assessment of pathological conditions, in particular autoimmune and/or inflammatory disorders. In particular, the inventors found out that a reliable assessment of these pathological conditions is possible by measuring IFI16 within an extracellular liquid sample from an individual, i.e. no tissue and no biopsy sample is required in diagnosis of the disease when using extracellular IFI16 protein as marker. Even more unexpectedly it was found that an increased level of extracellular IFI16 as measured from bodily fluid of an individual is associated with autoimmune or inflammatory disorders.

On the basis of the surprising result that IFI16 is significantly present in body fluids from SSc, SLE, SjS and rheumatoid arthritis (RA) patients, while it is only barely detectable in body fluids from healthy subjects, a particular usefulness as diagnostic tool for autoimmune disorders is assigned to the method according to the invention.

A specific role in the onset of inflammation has been assigned to IFI16 that, when overexpressed in endothelial cells, upregulates the expression of several proinflammatory cytokines and is involved in TNF-α and IFN signaling.

Thus the detection of IFI16 in the body fluid of patients by means of the method according to the invention is also particularly important with respect to inflammatory diseases, including autoimmune disorders and possibly to bacterial and viral infectious diseases (AIDS, meningitis, HCV infections), allergies, transplant reactions, cardiovascular and tumour diseases and so on. Furthermore, these are important for the determination of the response reaction with patients under treatment with inflammatory cytokines (e.g. interferon-α).

The detection or the quantification of IFI16 in a sample of a body fluid of a patient allows conclusions to be drawn about some clinical features of the patient.

Methods for obtaining the samples mentioned are known to the person skilled in the art. Optionally, the method according to the invention, moreover, comprises one or several washing steps prior to or after each method step. These washing steps serve the minimization of unspecific reactions (false positive or false negative detection) and can improve the sensitivity of the method. Suitable washing buffers and their composition are, in principle, known to the person skilled in the art. Physiological buffer solutions are preferred.

A preferred embodiment of the method according to the invention, moreover, comprises step (a′) or (a″) prior to contacting with the first receptor: (a′) labelling of the proteins contained in the sample; or (a″) labelling of the first receptor.

The proteins contained in the sample and/or the first receptor can, for example, be labelled chemically, e.g. by coupling of labelled chemical groups or markers to free amino groups of cysteines contained in the proteins. Examples of such marked chemical groups are groups containing special, detectable radioisotopes. For example fluorescent dyes can also serve as markers. A further example of appropriate markers are nucleic acids. The presence of proteins or receptors in a sample which are labelled in such a way can then be detected with suitable primers in a polymerase chain reaction (PCR).

Furthermore, it is possible to label proteins physiologically, i.e. by the metabolic integration of labelled molecules. For this purpose, cells are, for example, incubated with radioactively labelled metabolites. Proteins originating from the biosynthesis of these cells during this incubation period and in which the labelled metabolites were integrated are marked. This method is e.g. suitable to label antibodies secreted by cells which produce antibodies.

In a further preferred embodiment of the method according to the invention, the receptor is immobilised on a surface prior to contacting with a sample suspected to contain IFI16.

According to an alternative embodiment of the method of the invention, the receptor is immobilised on a surface after contacting with a sample suspected to contain IFI16.

Receptors can be immobilised in various way. The appropriate method depends on various factors, such as e.g. the type of receptor or the material of the surface. An immobilisation can take place covalently or by adsorption. According to a preferred embodiment of the method according to the invention, the receptors are proteins, particularly preferred antibodies. Also preferred is the use of peptides or organic molecules as receptors.

For the immobilisation of receptors which are proteins, methods are described in which the receptors are immobilised directly on a surface by means of passive adsorption. Normally, an appropriate surface consists of a polymer plastic material (e.g. polystyrene, polyvinyl, latex) and e.g. in form of microtitre plates or multi-well plates, membranes or spheric “beads” (cross-linked polymers in particle form) are used for this purpose (Lowman, Annu. Rev. Biophys. Biomol. Struct. 26 (1997), 401-24).

In a further preferred embodiment of the method according to the invention, the material of the surface is selected from the group consisting of sepharose, latex, glass, polystyrene, polyvinyl, nitrocellulose and silicon. Further preferred, the surface in the method according to the invention is a membrane, a bead, a chip or a plate.

Examples of beads are sepharose beads or latex beads, to which, optionally, ligands are bound, which promote the immobilisation of the receptors to the surface. Such ligands are, for example, protein A or protein G which promote a binding of antibodies to a surface via the Fc part of the antibody. The binding of the receptor to a carrier material can also be achieved by a covalent chemical coupling reaction (e.g. hydrazide coupling). Another example of the immobilisation of the receptors to the surface by means of ligands is the use of biotin and avidin or streptavidin.

Examples of chips are silicon plates onto which a plurality of different or the same receptors can be immobilised systematically. This allows the analysis of a plurality of different parameters in a sample or the analysis of a plurality of different samples as to one or several parameters, e.g. identification and/or quantification of IFI16 or fragments of this protein in different tissue samples, samples of body fluid or samples of cell culture supernatants.

Examples of the plates mentioned are microtitre plates or multi-well plates. Preferably, these have 6, 12, 24, 48, 96, 128, 356, 1024 or more wells. In Example 1, a method is described wherein 96-well plates are used.

According to a further preferred embodiment of the method, it further comprises step (b′) prior to the step of detection of a specific binding: (b′) Precipitating the beads with the complexes of the first receptor and IFI16.

Beads can be precipitated from a sample e.g. in a gravimetric manner. This can be accelerated, for example, by centrifugation. Appropriate methods are known to the person skilled in the art, amongst others from Rehm, Der Experimentator: Proteinbiochemie/Proteomics, Spektrum Akademischer Verlag, 2002.

In a further preferred embodiment of the method according to the invention, the detection of the specific binding between a receptor and IFI16 comprises a gel electrophoretic separation of the sample and, optionally, furthermore, a Western blot analysis. Appropriate methods are known to the skilled person, among others from Rehm, loc. cit. Furthermore, a corresponding method is described in Examples 2 and 4.

The method according to the invention is preferably carried out automatically. This is possible, amongst others, by the use of pipetting robots and for an automated analysis of optimised processes.

Furthermore, the invention refers to the use of body fluids or a sample of a cell culture supernatant, as defined above, for the detection of extracellular IFI16. Preferably, the positive detection is indicative for the presence of a pathological condition, in particular an inflammatory and/or autoimmune disease.

Further, the method of the invention also comprises the determination of at least one additional diagnostic marker, e.g. a diagnostic marker indicative of an autoimmune and/or inflammatory disorder. In a preferred embodiment, the at least one additional diagnostic marker is an anti-IFI16-autoantibody. The determination of several diagnostic markers may be carried out in parallel on a single sample or different aliquots of a single sample or on different samples. The concentration of the diagnostic markers are then interpreted independently, e.g. using an individual cut-off value for each marker, or they are combined for interpretation.

Finally, the present invention refers to a reagent kit for diagnostic use comprising:
(i) at least one receptor, which specifically binds to IFI16, and
(ii) further kit components, e.g. buffers, salts, reagents and/or instructions for use.

Preferred embodiments of reagent kits comprise two receptors, wherein one receptor is a detectable receptor and one receptor is an immobilized or an immobilizable receptor.

Further, the present invention shall by explained in more detail by the following figures and examples without being limited thereto.

### FIGURES

FIG. 1: Schematic representation of a IFI16 sandwich ELISA.

FIG. 2: Sensitivity and linearity of IFI16 sandwich ELISA. ELISA microtitre plates were coated with a polyclonal rabbit-anti-IFI16 antibody. Subsequently, the plates were washed with PBS-Triton (PBS-T; 0.25% Triton X100 in PBS) and for 30 minutes, free binding sites were saturated with PBS-T/BSA 3% (PBS-TB) at 37°. After washing with PBS-T, an incubation followed (1 h) with purified 6His-IFI16 protein, diluted in 5% FCS in PBS-T was used as standard. BSA served as negative control. The samples were washed 3 times with PBS-T, in each case monoclonal mouse anti-IFI16 antibody was added and incubated for 1 h at room temperature. After washing four times with PBS-T, an incubation followed (1 h, room temperature) with 100 μl, in each case, of HRP-conjugated anti-mouse antibody diluted in PBS-TB. After 3 washing steps, the IFI16 protein/antibody complex was visualised by incubation with tetramethylbenzidine (TMB) and stopped with Stop Solution. The absorption was measured at 450 nm in the micro plate reader. The determination of the concentration was carried out using the standard curve of FIG. 2 for which increasing concentrations of purified 6His-IFI16 were used. The linearity of the measurement is indicated for a range of 1 to 15.6 ng/ml.

FIG.3: Measuring circulating IFI16 in autoimmune patients and healthy subjects.

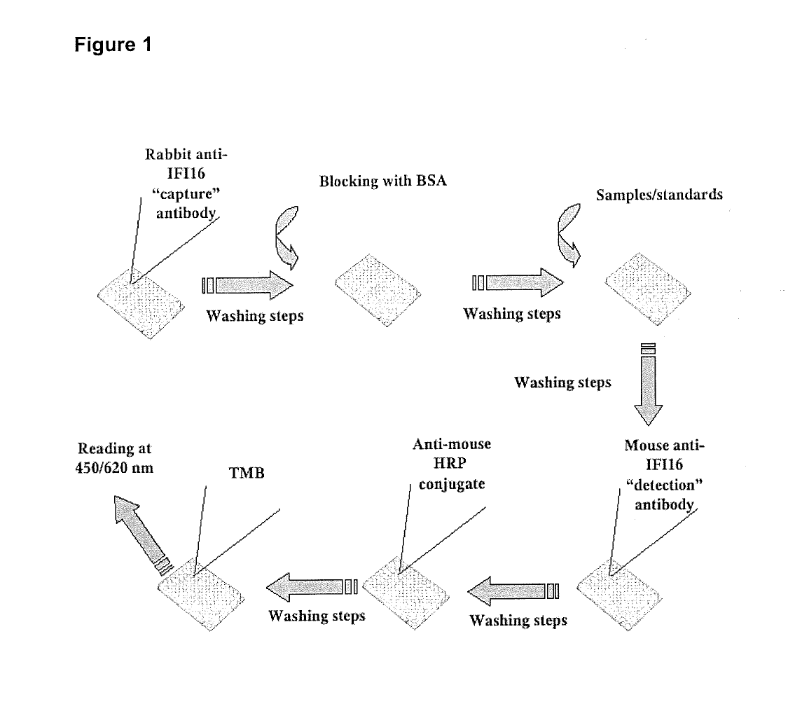

The concentration of circulating IFI16 in sera was determined by means of ELISA in patients suffering from SSc (99), SLE (30), SjS (20), RA (30) and patients with hepatitis C virus infection (HCV, 30) in healthy subjects (CTRLS, 54).

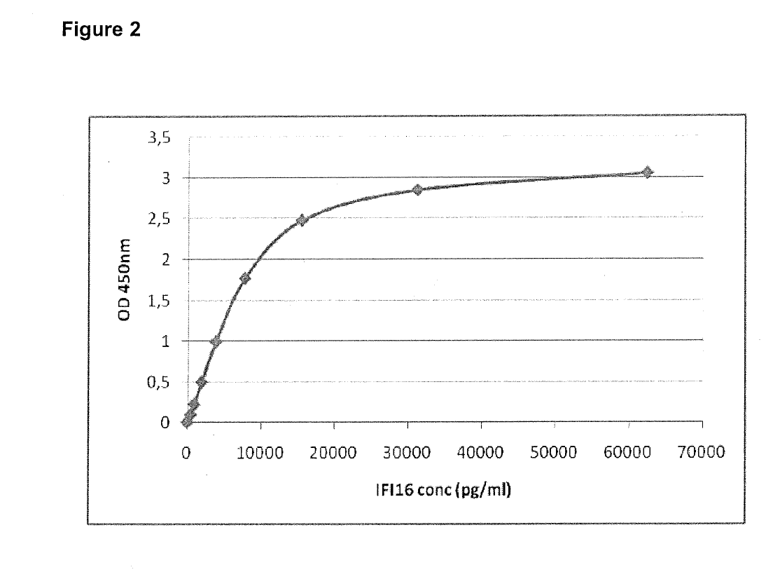

ELISA microtitre plates were coated with a polyclonal rabbit-anti-IFI16 antibody. Subsequently, the plates were washed with PBS-Triton (PBS-T; 0.25% Triton X100 in PBS) and for 30 minutes, free binding sites were saturated with PBS-T/BSA 3% (PBS-TB) at 37°. After washing with PBS-T, an incubation followed (1 h) with 5 μl of different sera samples in a final volume of 100 μl. Purified 6His-IFI16 protein, diluted in 5% FCS in PBS-T was used as standard. BSA served as negative control. The samples were washed 3 times with PBS-T, in each case monoclonal mouse anti-IFI16 antibody was added and incubated for 1 h at room temperature. After washing four times with PBS-T, an incubation followed (1 h, room temperature) with 100 μl, in each case, of HRP-conjugated anti-mouse antibody diluted in PBS-TB. After 3 washing steps, the IFI16 protein/antibody complex was visualised by incubation with tetramethylbenzidine (TMB) and stopped with Stop Solution. The absorption was measured at 450 nm in the micro plate reader. The determination of the concentration was carried out using the standard curve of FIG. 2 for which increasing concentrations of purified 6His-IFI16 were used. The linearity of the measurement ranged from 20 to 400 ng/ml IFI16 in the sera. Sera with a concentration outside the linearity range (<20 ng/ml or >400 ng/ml) are plotted as having 0 ng/ml or 400 ng/ml respectively.

The IFI16 serum protein was detectable in a fraction of patients sera (ranging from 54% to 84%), while IFI16 serum concentration was below the detection limit of the assay in all the healthy subjects.

FIG.4: Identification of extracellular IFI16 in cell supernatants. Human keratinocytes were exposed to UVB irradiation at a dose of 200, 400 or 800 J/m2 (UV 200, UV 400 or UV 800 respectively) or mock irradiated (NT) and then incubated for 16 or 24 hours (16 h or 24 h respectively). Supernatants were collected and extracellular proteins precipitated by TCA as described in Example 2. Immunoblotting analysis using anti-IFI16 polyclonal antibodies revealed the presence of extracellular IFI16 in supernatants of cells exposed to UVB irradiation doses of 400 and 800 J/m². Total cellular proteins extracted from human keratinocytes (TE), expressing intracellular IFI16, were used as a positive control for IFI16 immunoblotting.

FIG. 5: Sensitivity and linearity of IFI16 sandwich ELISA with improved linearity.

ELISA microlitre plates were coated with a polyclonal rabbit-anti-IFI16 antibody. Subsequently, the plates were washed and free binding sites were saturated with PBS/0.05% Tween-20/3% BSA (PBS-TB) at room temperature for 1 hour. After washing, an incubation followed (1 h, room temperature) with purified 6His-IFI16 protein, diluted in 5% FBS in PBS/0.05% Tween-20/1% BSA (PBS-TD), that was used as standard. BSA served as negative control. The samples were washed and in each case monoclonal mouse anti-IFI16 antibody was added and incubated for 1 h at room temperature. After washing, an incubation followed (1 h, room temperature) with HRP-conjugated anti-mouse antibody. After washing, the IFI16 protein/antibody complex was visualized by incubation with tetramethylbenzidine (TMB) and stopped with Stop Solution. The absorption was measured at 450 nm in the micro plate reader. The determination of the concentration was carried out using the standard curve of FIG. 5 for which increasing concentrations of purified 6His-IFI16 were used. The linearity of the measurement is indicated for a range of 1 to 32 ng/ml.

FIG. 6: Measuring circulating IFI16 in autoimmune patients and healthy subjects using IFI16 ELISA with improved linearity.

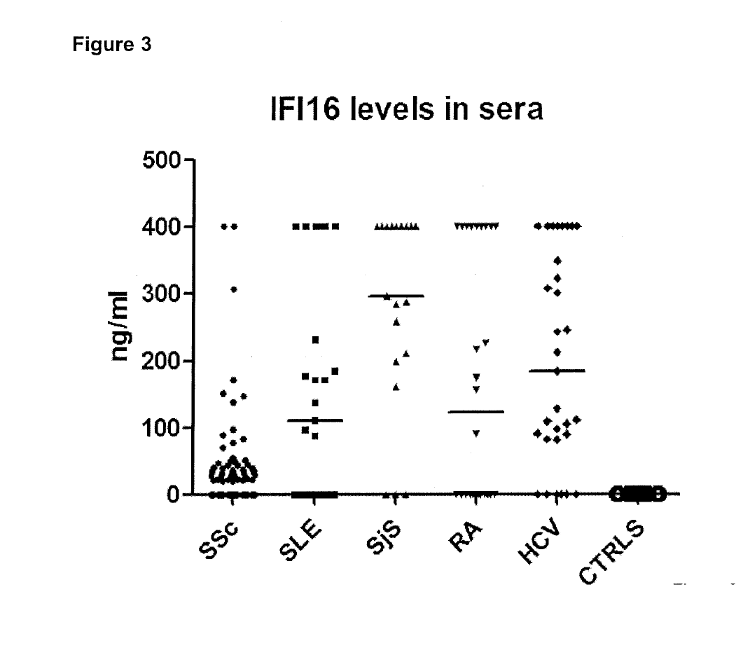

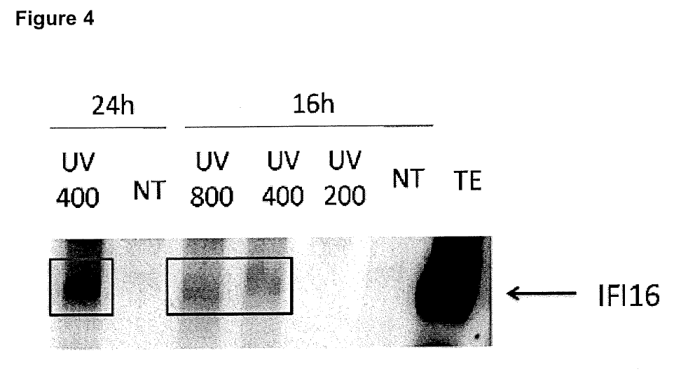

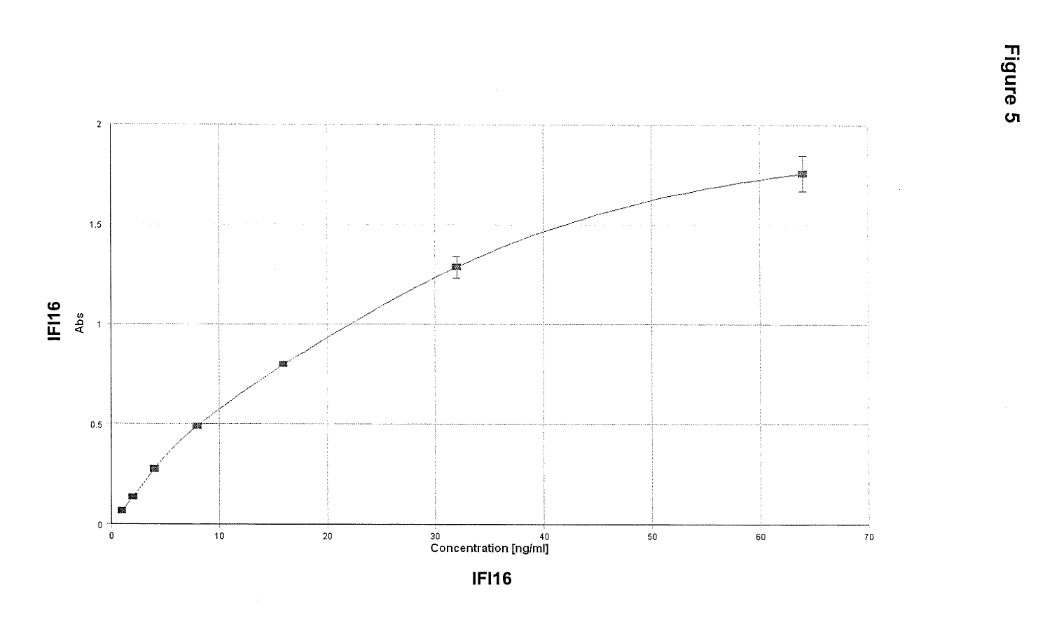

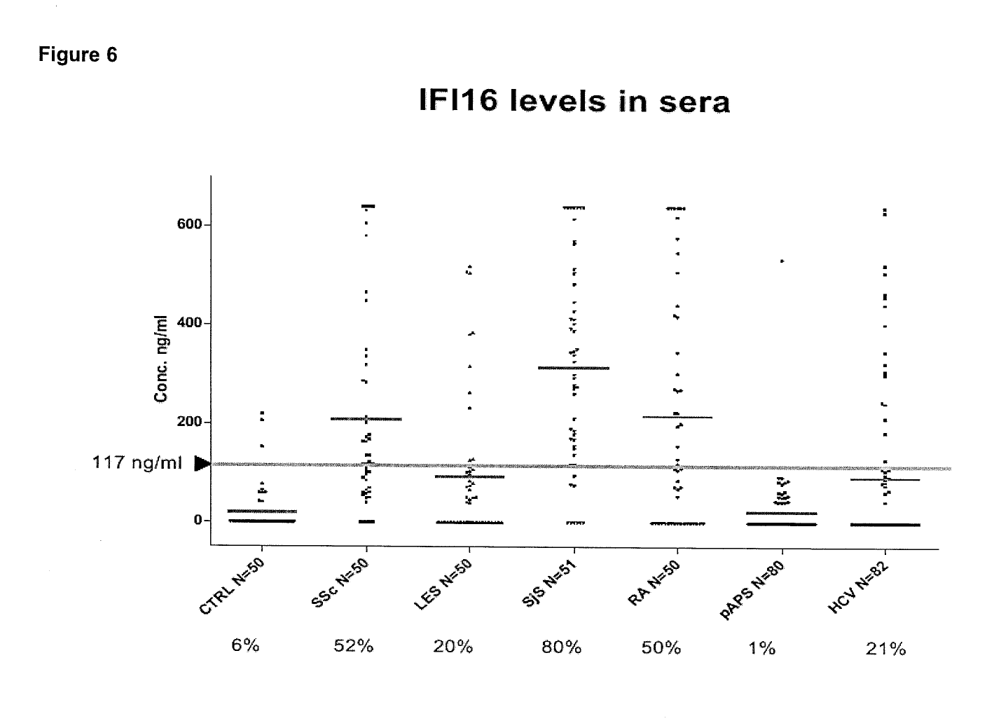

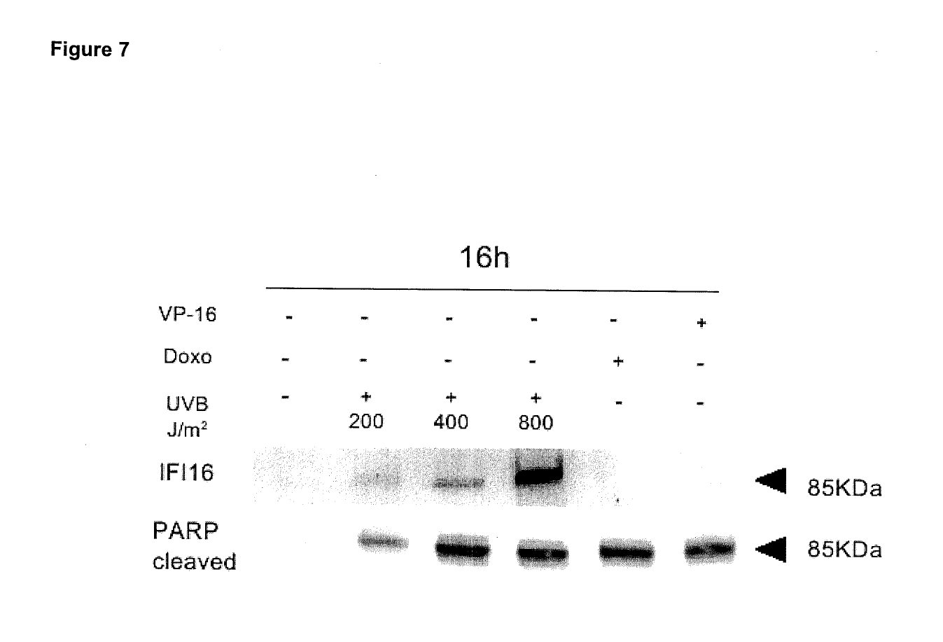

The concentration of circulating IFI16 in sera was determined by means of ELISA in patients suffering from SSc (50), SLE (50), SjS (51), RA (50), anti-phospholipid syndrome (pAPS, 80) and patients with hepatitis C virus infection (HCV, 82) and in healthy subjects (CTRL, 50). The cohorts tested represent different patients from those tested in FIG. 3.

ELISA microtitre plates were coated with a polyclonal rabbit-anti-IFI16 antibody. Subsequently, the plates were washed and free binding sites were saturated with PBS/0.05% Tween-20/3% BSA (PBS-TB) at room temperature for 1 hour. After washing, an incubation followed (1 h, room temperature) with 5 μl of different sera samples in a final volume of 100 μl of PBS/0.05% Tween-20/1% BSA (PBS-TD). Purified 6His-IFI16 protein, diluted in 5% FBS in PBS-TD was used as standard. BSA served as negative control. The samples were washed and in each case monoclonal mouse anti-IFI16 antibody was added and incubated for 1 h at room temperature. After washing, an incubation followed (1 h, room temperature) with HRP-conjugated anti-mouse antibody diluted in PBS-TD. After washing, the IFI16 protein/antibody complex was visualised by incubation with tetramethylbenzidine (TMB) and stopped with Stop Solution. The absorption was measured at 450 nm in the micro plate reader. The determination of the concentration was carried out using the standard curve of FIG. 5 for which increasing concentrations of purified 6His-IFI16 were used. The linearity of the measurement ranged from 20 to 640 ng/ml IFI16 in the sera. Sera with a concentration outside the linearity range (<20 ng/ml or >640 ng/ml) are plotted as having 0.1 ng/ml or 640 ng/ml respectively. The single grey horizontal lines represent the mean IFI16 concentrations for each group.

A cut-off value for IFI16 positivity was set at 95° percentile of control population (117 ng/ml), and is represented by the light grey continuous horizontal line. The numbers below the X axis represent the percentage of patients with IFI16 serum concentrations higher than the cut-off level in each group. The IFI16 serum protein was detectable at level higher than the cut-off in a fraction of SSc, SLE, SjS, RA and HCV patients sera ranging from 20% to 80%, while only in 6% the healthy subjects. Only 1% of patients suffering from pAPS were positive for circulating IFI16.

FIG. 7: Identification of extracellular IFI16 in supernatants of cells undergoing cell death.

Human Keratinocyte monolayers were UVB-irradiated at different doses (200, 400, and 800 J/m² respectively), treated with 2 μM Doxorubicin (Doxo) and 80 μM Etoposide (VP-16), or left untreated. At 16 h after treatment, the supernatant was collected and separated for determination of extracellular IFI16, while the remaining cells were lysed for determination of the intracellular cleaved form of PARP (determination of undergoing cell death). Collected supernatants were concentrated with TCA 25% as described in Example 4. An equal amount of total cellular protein per sample and an equal volume of concentrated supernatants were fractionated on SDS-PAGE (7.5% of NEXT GEL Amresco, OH, USA) and transferred to a nitrocellulose membrane (Biorad, CA, USA). Immunoblotting analysis using anti-IFI16 polyclonal antibodies revealed the presence of extracellular IFI16 in supernatants of cells exposed to UVB irradiation doses of 400 and 800 J/m². This phenomenon is not generally associated with cell damage, because it was not observed in keratinocytes undergoing chemically-induced cell death upon exposure to pharmacological cytotoxic drugs as Doxorubicin and Etoposide (as demonstrated by PARP cleavage, a recognized marker of necrotic and apoptotic cell death (Cepeda V. et al., Recent Pat Anticancer

Drug Discov. 2006 January;1(1):39-53)).

### EXAMPLE 1

### IFI16 ELISA

The following buffers were used for the IFI16 ELISA developed: PBS-T (0.25% Triton X100 in PBS); and PBS-TB (0.25% Triton X100 and 3% BSA in PBS).

96-well ELISA plates (Nunc-Maxisorb Plates) were coated with 100 μl/well anti-IFI16 polyclonal antibody (incubation at 4° C. for 16 h). The plates were washed with PBS-T and blocked with PBS-TB for at least 30 min at room temperature. The wells were aspirated and incubated as duplicates for 1 h at 37° C. temperature with 100 μl of the standard (6His-IFI16), diluted in a 5% FBS in PBS-TB, or with 100 μl of a sample at a suitable dilution (diluted with PBS-TB), respectively. The wells were washed four times with PBS-T and incubated for 1 h at 37° C. with 100 μl of a monoclonal mouse antibody against IFI16, diluted in PBS-TB. Subsequently, the wells were washed 3 times with PBS-T and incubated with 100 μl of a peroxidase (HRP) which is conjugated to a rabbit anti-mouse antibody (GE HealthCare, USA), diluted 1:500 in PBS-TB, for 1 h at 37° C. Subsequently, the wells were washed 3 times with PBS-T and were incubated with 100 μl tetra-methylbenzidine (SureBlue-TMB, KPL, USA) and then stopped with 100 μl of stop solution (TMB StopSolution, KPL, USA). The absorption was determined at 450 nm in a micro plate reader (Tecan), with 620 nm as a reference. The concentration of IFI16 in the sample was calculated by means of the standard curve. The method showed a linearity of 1 to 15.6 ng/ml of IFI16/well. The variability of the results in the different assays was 9.7% (inter-assay CV %=9.7%).

### EXAMPLE 2

### Western Blot Analysis of IFI16 in Cell Supernatants

Supernatants of human primary keratinocytes cultured in serum-free medium (Epilife, Cascade Biologics, USA) were subjected to precipitation with trichloroacetic acid (TCA). Precipitated protein were analyzed by immunoblotting.

Cells were cultured in serum-free medium (Epilife, Cascade Biologics, USA) and then exposed to different doses (from 200 to 800 J/m²) of ultraviolet B irradiation (UVB) or mock-irradiated. 16 and 24 hours after irradiation, supernatants were collected and centrifuged at 5000 g for 10 minutes to remove cellular debris. TCA was then added to supernatants at a final concentration of 25% v/v, samples were incubated 10 min on ice and centrifuged at 4° at 14000 g for 10 min. The protein pellet was washed 3 times with 100% acetone, air dried and resuspended in Laemmli Sample Buffer. Following denaturation at 95° for 5 min, the samples were loaded on 7.5% polyacrylamide gel and subjected to gel electrophoresis.

Migrated proteins were transferred to nitrocellulose. The membrane was blocked in TBS-5% BSA and extracellular IFI16 was detected by membrane incubation overnight at 4° C. with anti-IFI16 rabbit polyclonal antibody. After 3 washes with TBS-0.05%-Tween20 (TBS-T), the membrane was incubated with a HRP-conjugated anti-rabbit secondary antibody (GE Healthcare, USA) for 1 hour at room temperature. After washing with TBS-T, the membrane was incubated with ECL (GE HealtCare) and the chemiluminescent signals acquired by GelDoc image analyzer (BioRad, USA).

### EXAMPLE 3

### IFI16 ELISA with Improved Linearity

The following buffers were used for the IFI16 ELISA developed: PBS-TB (0.05% Tween-20 and 3% BSA in PBS) and PBS-TD (0.05% Tween-20 and 1% BSA in PBS).

96-well ELISA plates (Nunc-Maxisorb Plates) were coated with 100 μl/well anti-IFI16 polyclonal antiantibody (incubation at 4° C. for 16 h). The plates were washed with the wash buffer (Wash Solution Concentrate, KPL, USA) and blocked with PBS-TB for at least 1 hour at room temperature. The wells were washed and incubated as duplicates for 1 h at room temperature with 100 μl of the standard (6His-IFI16), diluted in a 5% FBS in PBS-TD, or with 100 μl of a sample at a suitable dilution (diluted with PBS-TD), respectively. The wells were washed and incubated for 1 h at room temperature with 100 μl of a monoclonal mouse antibody against IFI16, diluted in PBS-TD. Subsequently, the wells were washed and incubated with 100 μl of a peroxidase (HRP) which is conjugated to a rabbit anti-mouse antibody (GE HealthCare, USA), diluted 1:500 in PBS-TD, for 1 h at room temperature. Subsequently, the wells were washed and incubated with 100 μl tetramethylbenzidine (SureBlue-TMB, KPL, USA) and then stopped with 100 μl of stop solution (0.6N H₂SO₄). The absorption was determined at 450 nm in a micro plate reader (Tecan), with 620 nm as a reference. The concentration of IFI16 in the sample was calculated by means of the standard curve. The method showed a linearity of 1 to 32 ng/ml of IFI16/well. A cut-off value was set at the 95° percentile of the control population. Subjects displaying IFI16 concentrations higher than the cut-off value were considered positive for the presence of circulating IFI16.

### EXAMPLE 4

### Western Blot Analysis of IFI16 in Supernatants of Cells Undergoing Cell Death

Supernatants of human primary keratinocytes cultured in serum-free medium (Epilife, Cascade Biologics, USA) were subjected to precipitation with trichloroacetic acid (TCA). Precipitated protein were analyzed by immunoblotting.

Cells were cultured in serum-free medium (Epilife, Cascade Biologics, USA) and then exposed to different doses (from 200 to 800 J/m²) of ultraviolet B irradiation (UVB) or treated with 2 μM Doxorubicin (Doxo) or 80 μM Etoposide (VP-16) or mock-irradiated. At 16 hours after treatement, supernatants were collected and centrifuged at 5000 g for 10 minutes to remove cellular debris. TCA was then added to supernatants at a final concentration of 25% v/v, samples were incubated 10 min on ice and centrifuged at 4° at 14000 g for 10 min. The protein pellet was washed 3 times with 100% acetone, air dried and resuspended in Laemmli Sample Buffer.

As control of the undergoing cell death induced by the treatment with UVB-irradiation and Doxo or VP-16, also the intracellular PARP cleavage was determined in the exposed keratinocyte. For this proposed the Adherent cells were lysed in RIPA buffer (50 mM Tris-cl pH 7.4, 150 mM NaCl, 1% NP40, 0.25% Na-deoxycholate, 1 mM PMSF, 1X complete mini protease inhibitor cocktail (Roche), 1X phosphatase inhibitor cocktail (Pierce)).

Following denaturation at 95° for 5 min, the samples were loaded on 7.5% polyacrylamide gel and subjected to gel electrophoresis.

Migrated proteins were transferred to nitrocellulose. The membranes were blocked in TBS/0.05% Tween20/5% BSA and extracellular IFI16 was detected by incubation of the membrane bearing surnatants samples with anti-IFI16 mouse monoclonal antibody (clone 1G7, Santa Cruz, Calif., USA). The intracellular cleaved form of PARP was detected by incubation of the membrane bearing cell extract samples by rabbit anti-PARP cleaved antibody (GTX24830, GeneTex, CA, USA). After 3 washes with TBS/0.05% Tween20 (TBS-T), the membranes were incubated with a HRP-conjugated anti-mouse or anti-rabbit secondary antibody (GE Healthcare, USA) respectively for 1 hour at room temperature. After washing with TBS-T, the membranes were incubated with ECL (GE HealtCare) and the chemiluminescent signals acquired by GelDoc image analyzer (BioRad, USA).

1. An in vitro method for determining extracellular interferon inducible pro-tein 16 (IFI16) in a sample.

2. The method of claim 1, wherein the sample is a body fluid sample, e.g., blood, plasma or serum, or a supernatant of a tissue sample or a supernatant of a cell culture sample.

3. The method of claim 1, wherein the determination comprises:
(a) contacting the sample with at least one receptor, which specifically binds to IFI16, and
(b) detecting the specific binding of the receptor to IFI16.

4. The method of claim 3, wherein the sample is contacted with at least two receptors, which specifically bind to IFI16, wherein one of the re-ceptors is a detectable receptor and the other receptor is immobilized on a solid phase or carries a solid-phase binding group.

5. The method of claim 3, wherein at least one receptor is an anti-body or an antigen-binding fragment thereof.

6. The method of claim 4, wherein the detectable receptor carries a detectable labelling group, e.g. an enzymatic, fluorescent, radioactive or nucleic acid labelling group.

7. The method of claim 1, wherein the sample is a human sample.

8. The method of claim 1, wherein the presence and/or an increased amount of extracellular IFI16 is indicative for a pathologic condition.

9. The method of claim 1, wherein the presence and/or an increased amount of extracellular IFI16 is indicative of an autoimmune and/or inflammatory disorder.

10. The method of claim 9, wherein the autoimmune disorder is selected from Systemic Sclerosis (SSc), Systemic Lupus Erythematosus (SLE) and Sjägren's Syndrome (SjS).

11. The method of claim 9, wherein the disorder is selected from rheumat-oid arthritis.

12. The method of claim 1, wherein the presence and/or an increased amount of extracellular IFI16 is indicative of an infective dis-order.

13. The method of claim 12, wherein the infective disorder is HCV infection.

14. The method of claim 1, further comprising determining of at least one additional diagnostic marker.

15. The method of claim 14, wherein the at least one additional diagnostic marker is an anti-IFI16-autoantibody.
